(19) United States
(12) Patent Application Publication
(10) Pub. No.: US 2005/0074370 A1
(43) Pub. Date: Apr. 7, 2005

(54) Essence bottle wick

(76) Inventor: Tiao-Ho Yuan, Taipei City (TW)

Correspondence Address: PRO-TECHTOR INTERNATIONAL, 20775 Norada Court, Saratoga, CA, 95070-3018, US

(21) Appl. No.: 10/680,614

(22) Filed: Oct. 6, 2003

(51) Int. Cl. A61L 9/03

(52) U.S. Cl. 422/126

(57) ABSTRACT

A wick for an essence bottle, used in conjunction with an essence bottle, comprising a base, a heat-resistant element, an oil feeder, a separating grid, and a catalyst. The base is set on the essence bottle, having a chamber and a lower end with a periphery from which projecting points protrude, so that a gap between the base and the bottle opening remains. The heat-resistant element is placed inside the chamber of the base, having a through hole which allows oil vapor to pass through. The oil feeder has an elongated shape with an upper end that reaches into an opening in the heat-resistant element and a lower end that is immersed in oil in the essence bottle, absorbing oil from there. The separating grid and the catalyst are stacked on the heat-resistant element. The catalyst brings about catalysis of oil vapor. Due to the separating grid, the catalyst only absorbs oil vapor rather than oil from the heat-resistant element, so that, after igniting oil vapor at the catalyst, a small flame results, oil consumption is reduced and no wax is generated that clogs the catalyst.

### BACKGROUND OF THE INVENTION

1. Field of the Invention

The present invention relates to a wick for an essence bottle, particularly to a wick for an essence bottle which ensures a small flame and low oil consumption without clogging a catalyst.

2. Description of Related Art

The living environment of humans contains large quantities of bacteria, fungi, allergy causing particles and carcinogens. Fighting these harmful substance by drugs puts a strain on health and results in unwanted side effects. Burning essence, however, kills microbes while protecting health as well as the environment.

Since burning of essence generates ozone (O₃), leading to the disintegration of noxious particles in the air. Essence contains alcohol, and a burning wick releases anions, both of which have the effect of killing microbes. Catalysis of a scent is brought about by a wick. As shown in FIGS. 6 and 7, a conventional wick for burning essence comprises: a base 70, set on the opening of a bottle, enclosing a chamber 71 and has a lower end with a periphery carrying projecting points 72; a heat-resistant element 73 inside the chamber 71 of the base 70, having a lower side with an opening 74 and an upper side with a hole 75 reaching through to the opening 74; an oil feeder 76, having an upper end connected with the heat-resistant element 73 and a lower end immersed into oil within the bottle; a catalyst 77 on the upper side of the heat-resistant element 73, bringing about catalysis of oil vapor; a hole 78 in the catalyst 77, allowing fragrant material to be released; and a fixing element 79, shaped like a staple and put over the catalyst 77, with two ends clinching the base 70 on opposite lateral sides thereof, so that the heat-resistant element 73 and the catalyst 77 are firmly held on the base 70.

For using the wick, first the oil feeder 76 is inserted into the bottle, and the base 70 is set on the opening of the bottle. Then the oil feeder 76 will have been completely filled with oil. The heat-absorbing element 73 by capillarity serves a s a conduit for oil passing to the catalyst 77. When oil at the catalyst 77 is ignited, heat is transferred to the heat-resistant element 73, so that oil is released through the hole 75 of the heat-resistant element 73 and the hole 78 of the catalyst 77, feeding a continuous flame at the catalyst 77.

After a time span of burning, generated heat activates the catalyst 77. After extinguishing the flame, the activated catalyst 77 brings about catalysis of oil vapor.

Although a conventional wick works usefully, the following shortcomings still remain:

1. Since the catalyst 76 and the heat-resistant element 72 are joined to form a single body, the catalyst 77 absorbs a large quantity of oil.

2. On igniting oil, a large flame is generated, so that oil is wasted and surrounding air is polluted.

3. A large flame, generated on igniting oil, is potentially dangerous.

4. Burning oil creates wax, which easily blocks the catalyst.

5. A large flame is hard to extinguish by blowing or by putting a cap on the bottle, causing difficulties during usage.

### SUMMARY OF THE INVENTION

It is an object of the present invention to provide a wick for an essence bottle which absorbs only small quantities of oil.

Another object of the present invention is to provide a wick for an essence bottle which reduces oil consumption.

A further object of the present invention is to provide a wick for an essence bottle with a catalyst that is not easily clogged.

A further object of the present invention is to provide a wick for an essence bottle where extinguishing a flame is riskless.

The present invention can be more fully understood by reference to the following description and accompanying drawings.

### DETAILED DESCRIPTION OF THE PREFERRED EMBODIMENTS

The wick for an essence bottle of the present invention is used in conjunction with an essence bottle 60 containing essence and having a bottle opening 61. As shown in FIGS. 1-4, the wick for an essence bottle of the present invention comprises: a base 10, set on the bottle opening 61; a heat-resistant element 20 inside the base 10; an oil feeder 30, attached to the heat-resistant element 20 on a lower side thereof; a separating grid 40 on an upper side of the heat-resistant element 20; and a catalyst 50 on the separating grid 40.

The base 10 has an inner chamber 11 and a lower end with a periphery surrounded by a projecting points 12. The projecting points 12 ensure that, when the base 10 is set on the essence bottle 60, gaps remain, allowing air to enter the bottle. The heat-resistant element 20 is placed in the chamber 11 inside the base 10, having an opening 21 and a central hole 22 on the upper side, which reaches to the opening 21, allowing oil vapor to pass through. The oil feeder 30 has an elongated shape with an upper end that reaches into the opening 21 and a lower end that is immersed in oil in the essence bottle 60, absorbing oil from there. The heat-resistant element 20, the separating grid 40 and the catalyst 50 are stacked on each other. The catalyst 50 has an upper side with a central hole 51, allowing oil vapor to pass through. The separating grid 40 keeps the heat-resistant element 20 and the catalyst 50 apart, preventing the catalyst 50 from absorbing oil directly from the heat-resistant element.

The base 10 has an upper edge into which a plurality of broad incisions are cut to provide ventilation. A positioning cap 14 is put on the base, holding the separating grid 40 and the catalyst 50 on the heat-resistant element 20.

A metal mesh 42 is inserted between the heat-resistant element 20 and the separating grid 40, transmitting heat generated at the catalyst 50 to the heat-resistant element 20, so that oil absorbed by the heat-resistant element 20 from the oil feeder is rapidly vaporized, feeding a flame on the catalyst 50. The metal mesh 42 has a central hole 43, allowing oil vapor to pass through.

Referring to FIG. 3, for using the wick of the present invention, first the base 10 is set on the bottle opening 61, and the oil feeder 30 is inserted into the essence bottle 60, so that the oil feeder 30 absorbs oil contained therein. Oil continuously vaporizes, and oil vapor passes through the central hole 22 of the heat-resistant element 20, the metal mesh 42 and the separating grid 40, reaching the catalyst 50. When oil vapor at the catalyst 50 is ignited, heat passes from there the separating grid 40 and through the metal mesh 42 to the heat-resistant element 20, accelerating vaporization of oil.

After a time span, having reached a predetermined temperature, the catalyst 50 is activated. Extinguishing the flame at this time results in catalysis of oil vapor by the catalyst 50.

Referring to FIG. 4, for discontinuing usage of the wick of the present invention, a bottle cap 62 is put on the essence bottle 60, completely covering the bottle opening 61 and blocking air from entering the essence bottle 60. After some time, catalysis of oil vapor by the catalyst 50 stops.

The present invention offers the following advantages:

1. The catalyst 50, being separated from the heat-resistant element 20 by the separating grid 40 and the metal mesh 42, will not absorb oil from the heat-resistant element 20 in large quantities. After ignition, oil consumption and air pollution are low.

2. Due to low absorption oil from the heat-resistant element 20 by the catalyst 50, no large flame is generated and related risks are eliminated.

3. Only catalysis of oil vapor is performed by the catalyst 50, hence no wax is generated and the catalyst 50 will not be clogged.

4. Since the flame after igniting is small, extinguishing thereof is performed without difficulties.

Referring to FIG. 5, in a second embodiment of the present invention, the base 10 on the upper edge thereof carries a plurality of fixing elements 14a, which are shaped like strips and are slightly bent inwards, holding the separating grid 40 and the catalyst 50 in fixed positions on the heat-resistant element 20.

Referring to FIG. 6, in a third embodiment the present invention has a separating grid 40a, a catalyst 50a, and a positioning cap 14a. The separating grid 40a has an outer peripheral grid 44a, protecting the catalyst 50a from shock and resulting damage and allowing to fasten the positioning cap 14a on the catalyst 50a with ease.

Referring to FIG. 7, in a fourth embodiment the present invention has a heat resistant element 20b with a central hole 22b, a catalyst 50b with a central hole 51b, and a separating grid 40b with a peripheral grid 44b and an enlarged central hole 41b surrounded by an inner peripheral grid 45b. The heat resistant element 20b carries a projection 23b, which reaches into the central hole 45b. The central hole 45b has an outer diameter that is slightly smaller than the inner diamater of the central hole 51b of the catalyst 50b, allowing easy installation within the central hole 51b. While essence is burned, the projection 23b of the heat resistant element 20b has a temperature that is lower than the temperature of the catalyst 50b, so that vaporized essence emanating from the central hole 22b of the heat resistant element 20b will not easily be damaged.

While the invention has been described with reference to preferred embodiments thereof, it is to be understood that modifications or variations may be easily made without departing from the spirit of this invention which is defined by the appended claims.

### BRIEF DESCRIPTION OF THE DRAWINGS

FIG. 1 is a perspective view of the fastening assembly for an essence bottle of the present invention, with the fixing cap embodied as a mesh.

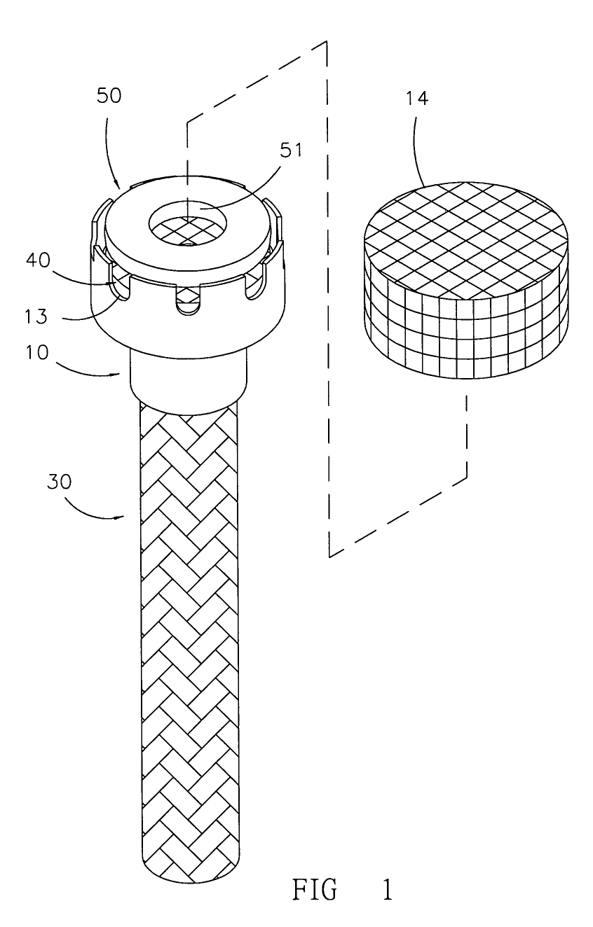

FIG. 2 is an exploded perspective view of the wick for an essence bottle of the present invention.

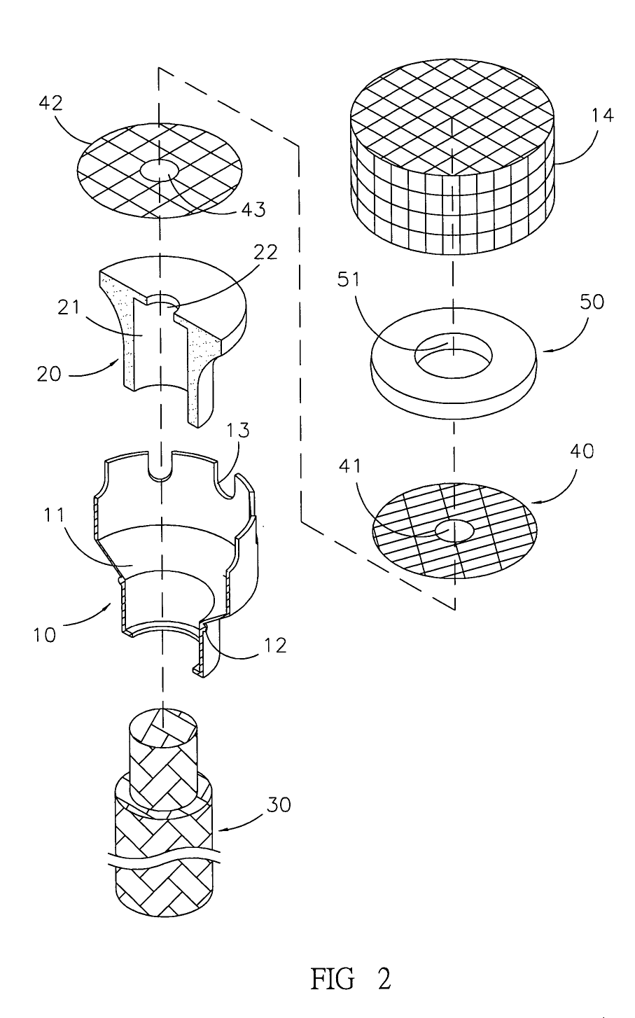

FIG. 3 is a sectional side view of the wick for an essence bottle of the present invention in conjunction with an essence bottle.

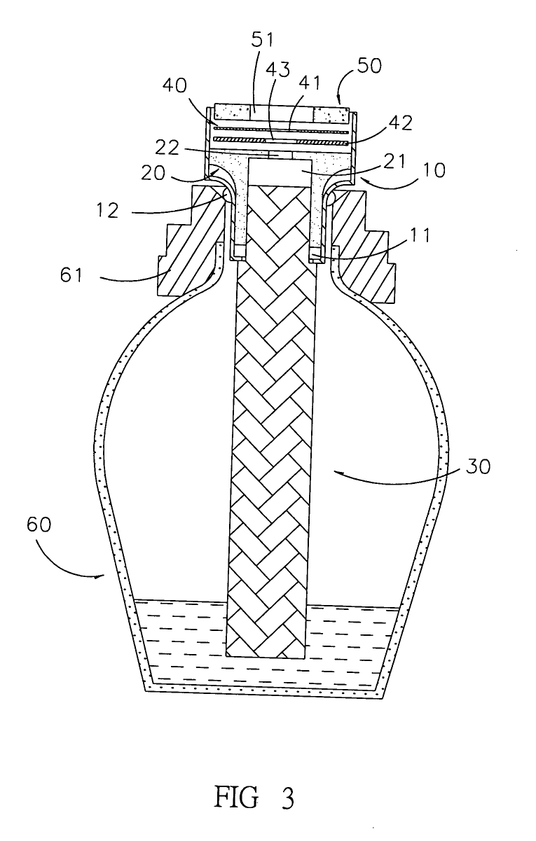

FIG. 4 is a side view of the present invention in conjunction with an essence bottle when covered by the bottle cap.

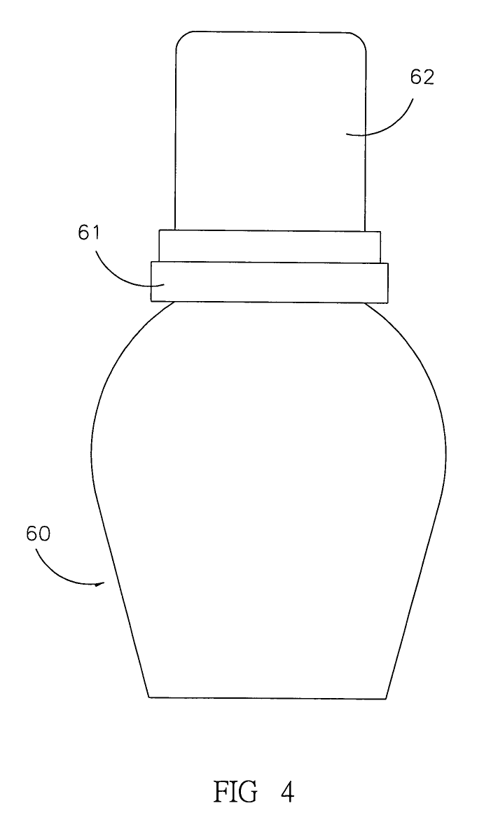

FIG. 5 is an exploded perspective view of the wick for an essence bottle of the present invention in the second embodiment.

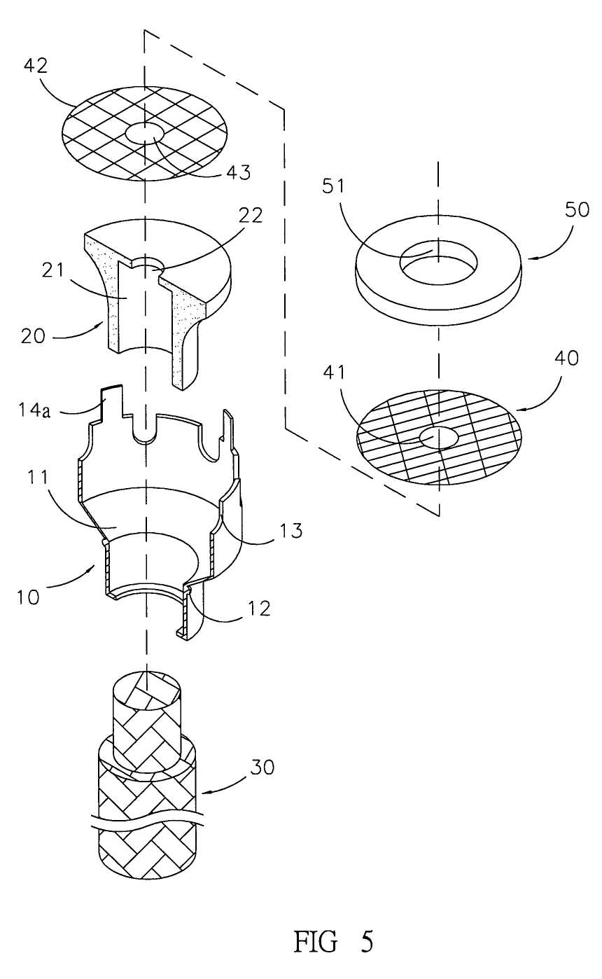

FIG. 6 is a sectional schematic view of the wick for an essence bottle of the present invention in the third embodiment.

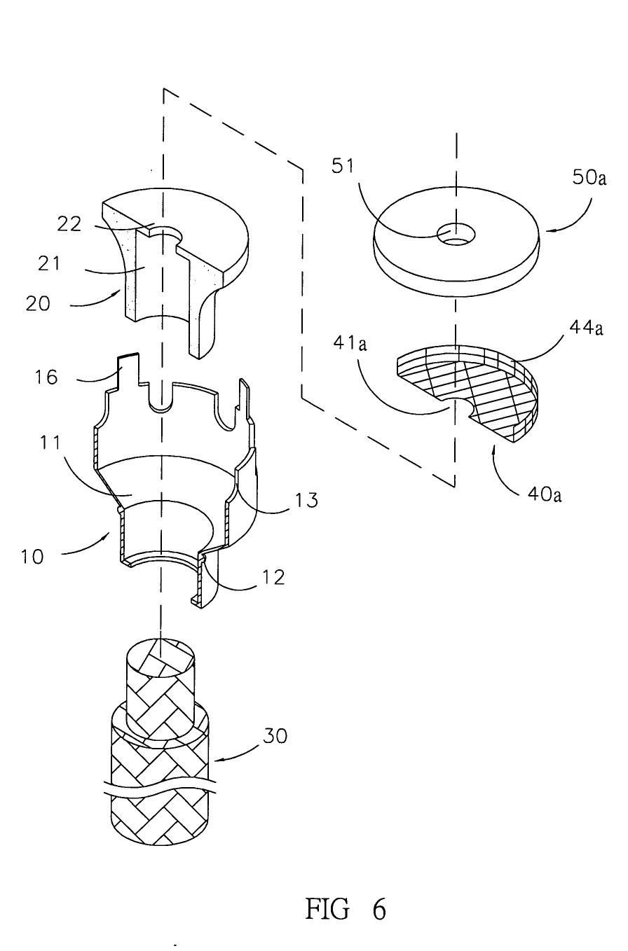

FIG. 7 is a sectional schematic view of the wick for an essence bottle of the present invention in the fourth embodiment.

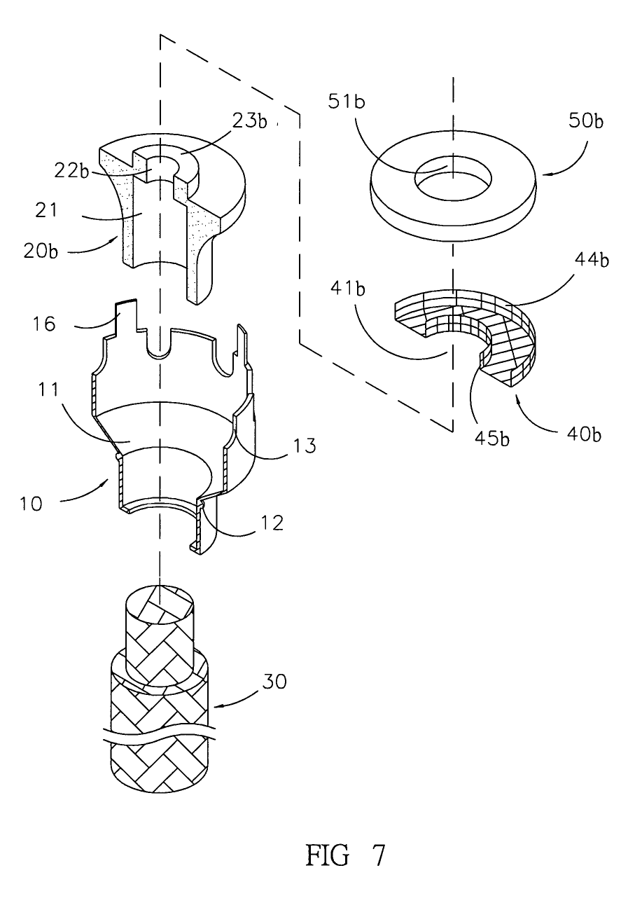

FIG. 8 is a perspective view of a conventional wick for an essence bottle.

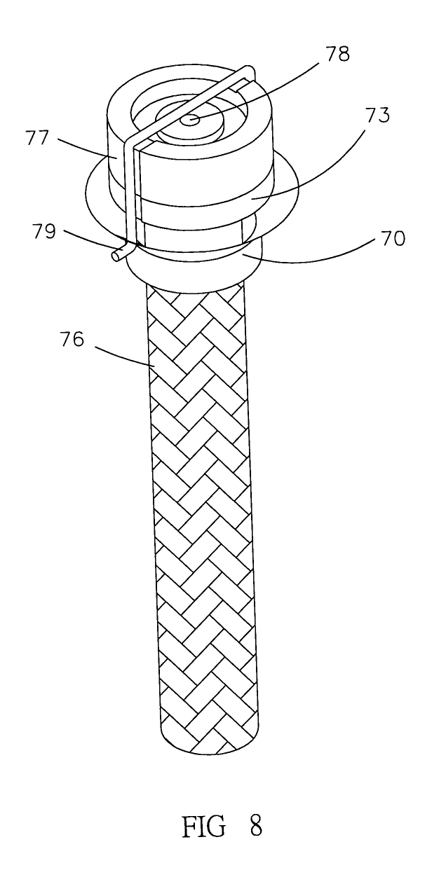

FIG. 9 is an enlarged sectional perspective view of a conventional wick for an essence bottle.

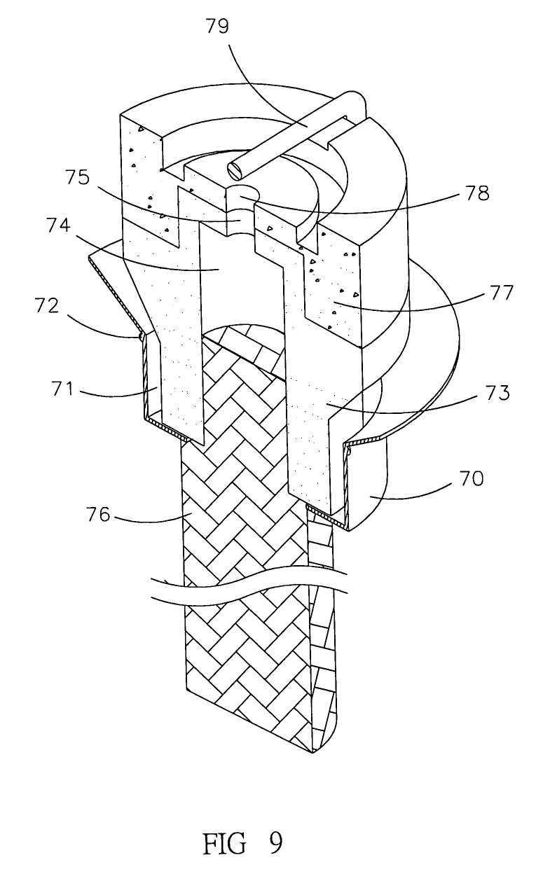

1. A wick for an essence bottle, used in conjunction with an essence bottle having a bottle opening on an upper end thereof, comprising:
a base, set on said bottle opening, having a chamber and a lower end with a periphery from which projecting points protrude, so that a gap remains between said base and said bottle opening;
a heat-resistant element, placed inside said chamber of said base, having a lower side with an opening and an upper side with at least one hole that reaches to said opening, allowing oil vapor to pass through;
an oil feeder, having an elongated shape with an upper end that reaches into said opening of said heat-resistant element and a lower end that is immersed in oil in said essence bottle, absorbing oil from there;
a separating grid, laid on said upper side of said heat-resistant element; and
a catalyst, laid on said separating grid bringing about catalysis of oil vapor;
wherein, due to said separating grid, said catalyst only absorbs oil vapor, so that, after igniting oil vapor at said catalyst, a small flame results, oil consumption is reduced and no wax is generated that clogs said catalyst.

2. The wick for an essence bottle according to claim 1, wherein said base has a plurality of broad incisions.

3. The wick for an essence bottle according to claim 1, wherein said base has a fixing element.

4. The wick for an essence bottle according to claim 3, wherein said fixing element is a mesh.

5. The wick for an essence bottle according to claim 3, wherein said fixing element is a fixing plate.

6. The wick for an essence bottle according to claim 1, wherein said heat-resistant element is made of porous ceramic material.

7. The wick for an essence bottle according to claim 1, wherein said separating grid is made of metal.

8. The wick for an essence bottle according to claim 1, wherein said separating grid has a hole.

9. The wick for an essence bottle according to claim 1, wherein a metal mesh is inserted between said heat-resistant element and said separating grid.

10. The wick for an essence bottle according to claim 1, wherein said separating grid has an outer peripheral grid.

11. The wick for an essence bottle according to claim 1, wherein said separating grid has an inner opening with an inner peripheral grid and an outer peripheral grid, with said heat-resistant element carrying a projection that extends into said inner opening of said separating grid and said catalyst being positioned between said inner grid and said outer grid.

12. The wick for an essence bottle according to claim 1, wherein said catalyst has a hole.

13. The wick for an essence bottle according to claim 9, wherein said metal mesh has a hole.
